(19) United States
(12) Patent Application Publication
(10) Pub. No.: US 2015/0114836 A1
(43) Pub. Date: Apr. 30, 2015

(54) Measurement Device with Reader and Disposable Probe

(71) Applicants: William Clark, Wexford, PA (US); Sung Kwon Cho, Pittsburgh, PA (US); Yuejun Zhao, Pittsburgh, PA (US); Timothy J. Syciarz, Pittsburgh, PA (US); R. Maxwell Flaherty, Auburndale, FL (US); J. Christopher Flaherty, Auburndale, FL (US)

(72) Inventors: William Clark, Wexford, PA (US); Sung Kwon Cho, Pittsburgh, PA (US); Yuejun Zhao, Pittsburgh, PA (US); Timothy J. Syciarz, Pittsburgh, PA (US); R. Maxwell Flaherty, Auburndale, FL (US); J. Christopher Flaherty, Auburndale, FL (US)

(21) Appl. No.: 14/343,384

(22) PCT Filed: Sep. 6, 2012
(86) PCT No.: PCT/US12/53905

§ 371 Date: Nov. 26, 2014

Related U.S. Application Data
(60) Provisional application No. 61/531,546, filed on Sep. 6, 2011.

(51) Int. Cl. G01N 27/30; A61B 5/1473; A61B 5/145

(52) U.S. Cl. CPC G01N 27/302; A61B 5/14539; A61B 5/1473
USPC 204/403.02

(57) ABSTRACT

A system for obtaining a pH measurement comprises a disposable probe and a reader. The disposable probe includes at least one indicating electrode and at least one reference electrode. The reader is configured to operably engage with the disposable probe and provide pH information of a sample.

### CROSS-REFERENCE TO RELATED APPLICATIONS

This application is related to Applicant's U.S. Provisional Application Ser. No. 61/531,546, entitled MEASUREMENT DEVICE WITH READER AND DISPOSABLE PROBE, by Clark et al, filed Sep. 6, 2011, the disclosures of which are incorporated herein by reference in their entirety.

### BACKGROUND OF THE INVENTION

The following information is provided to assist the reader to understand the technology described below and certain environments in which such technology can be used. The terms used herein are not intended to be limited to any particular narrow interpretation unless clearly stated otherwise in this document. References set forth herein may facilitate understanding of the technology or the background thereof. The disclosure of all references cited herein are incorporated by reference in their entirety.

A typical pH sensor based on potentiometric principles includes a reference electrolyte solution, an indicating electrode immersed in or in contact with an analyte solution (of which the pH is to be measured), a reference electrode immersed in the reference electrolyte solution, and measurement circuitry such as potentiometric circuitry in electrical connection with the reference electrode and the indicating electrode. The potentiometric circuitry measures the electrical difference between the indicating and reference electrodes. Ionic contact between the electrolyte solutions in which the indicating electrode and the reference electrodes are immersed provides electrical connection between the electrodes. The pH value of the sample or analyte electrolyte solution (which is proportional to concentration of the hydrogen ions in the sample electrolyte) is directly correlated with the potential difference developed at the indicating electrode following the Nernst equation.

In the above-described configuration, an important condition for correct measurement is that the electric potential difference built up in the reference electrode and the reference electrolyte is maintained constant such that the reading from the potentiometric circuitry solely represents the potential difference in the indicating electrode, that is, pH in the electrolyte solution. To meet this condition, a common arrangement is to have the reference electrode immersed in a saturated reference electrolyte solution, and to have a small “window” positioned between the saturated reference electrolyte solution and the sample or analyte electrolyte solution to provide ionic contact and thus an electrical connection between the saturated reference electrolyte solution and the sample or analyte electrolyte solution. The “window” is usually fabricated from a porous material such as a porous glass membrane, a hydrophilic porous polymer membrane, etc. Because of the porosity of the “window”, a non-negligible mass exchange occurs between the saturated reference electrolyte solution and the sample or analyte electrolyte solution, thereby causing cross-contamination in both solutions.

The dilution of the saturated reference electrolyte solution resulting from such contamination can be a significant problem since it changes the potential difference in the reference electrode. The contamination also deteriorates the stability of the pH sensor and shortens the lifetime of the pH sensor. As the dimensions of a pH sensor are reduced (for example, to very small, microlevel, microscale or smaller dimension), the problem is exacerbated because the volume of the saturated reference electrolyte solution is very small compared to the sample electrolyte solution. For example, for applications where a microscale or smaller pH sensor is implanted into a human body and is utilized to measure a physiological pH (for example, myocardial pH), the volume of the saturated reference electrolyte solution is extremely small compared to the volume of the myocardial tissue of which the pH is to be measured. At such a scale, the saturated reference electrolyte solution is diluted much more quickly than in a macro scale glass tube type pH sensor.

Another factor which affects the useful life of a pH sensor, such as a microscale pH sensor, is the durability of the reference electrode. In many instances, conductive material of the reference electrode is gradually dissolved and consumed into the saturated reference electrolyte solution. At some point during the dissolution and consumption of the reference electrode, the useful life of the pH sensor is terminated.

### SUMMARY

According to a first aspect of the inventive concepts, a system for obtaining a pH measurement comprises a reader and a disposable probe. The disposable probe comprises at least one indicating electrode and at least one reference electrode. The reader is configured to operably engage with the disposable probe and provide pH information of a sample. The system is constructed and arranged to provide the pH information based on potentiometric measurement of the sample solution including a measurement of at least two signals. A first signal is received from the at least one indicating electrode when the at least one indicating electrode is in contact with the sample. A second signal is received from the at least one reference electrode when the at least one reference electrode is in contact with a reference solution. The system may include one or more components manufactured in a MEMS or other automated process, such as a disposable probe or probe portion manufactured in a MEMS process.

The system may include a reservoir, such as a buffer solution reservoir positioned in the disposable probe or the reader. A barrier may be included to separate the reservoir from another component of the system, such as a separation to the at least one reference electrode. The barrier or a portion of the barrier may be configured to be removed or opened such as to allow flow of fluid. The barrier may be removed or opened through the application of a force, such as a force exerted by an operator of the system or by a component of the system such as a component activated by an electronics module of the system.

One or more fluidic channels may be included in the disposable probe and/or the reader. A liquid junction may be included, such as a virtual liquid junction positioned in a fluidic channel. Fluid such as reference solution fluid, sample fluid and/or other fluids may be moved or otherwise transported in the fluidic channels, such as via an automatic or manual pumping mechanism located in the reader. In one embodiment, a first fluidic channel is in fluid communication with a first pumping mechanism and a second fluidic channel is in fluid communication with a second pumping mechanism.

The disposable probe may include multiple portions, such as a first portion including the at least one reference electrode and a second portion including the at least one indicating electrode. A first disposable probe portion may operably engage with a first port of the reader while a second disposable probe portion engages a second port of the reader. The disposable probe may comprise a multi-layer construction, such as a construction including a substrate comprising glass, silicon and/or plastic.

The at least one indicating electrode may comprise an iridium oxide electrode. Two or more indicating electrodes may be included in the system. One or more indicating electrodes may be individually activated, such as through activation by an electronics module of the system. In one embodiment, a controllable orifice may be positioned over an indicating electrode. Indicating electrodes may be mounted to a substrate, such as via a mounting pad, such as a titanium mounting pad.

The at least one reference electrode may be configured to perform multiple measurements of one or more samples. Reference electrodes typically comprise silver-silver chloride electrodes and/or iridium oxide electrodes. One or more covers may surround one or more reference electrodes.

The reader is configured to operably engage one or more ends of the disposable probe via a port. The port is configured to electrically connect one or more electrical wires, traces or other conductors of the disposable probe to one or more electrical wires, traces or other conductors of the reader. The port may also connect one or more fluidic channels of the disposable probe to a corresponding one or more fluidic channels of the reader, such as to transport fluid such as buffer solution or other fluid to or from the reader from or to the disposable probe. In one embodiment, fluid is drawn into the reader such that sample fluid is drawn into the distal end of the disposable probe, such that the sample fluid covers a distally placed indicating electrode.

The reader typically includes an electronics module configured to perform one or more functions including but not limited to: store data; communicate with one or more external devices such as via wired or wireless communications; perform internal diagnostic checks; and combinations of these. The reader may be configured to display pH information as well as system information. System information typically includes but is not limited to: system readiness information; power levels; alert or alarm condition information; current status of disposable; and combinations of these.

The system may include one or more sensors, such as a sensor selected from the group consisting of: a temperature sensor; a humidity sensor; a pressure sensor; and combinations of these. Signals received by the one or more sensors may be used by the system to determine the pH information, such as in an algorithm that mathematically takes into account environmental conditions. The one or more sensors may be located in the reader, the disposable probe, or both.

The system may include two or more disposable probes.

According to a second aspect of the inventive concepts, a method of using a system for obtaining a pH measurement is disclosed. The system comprises a reader and a disposable probe. The disposable probe comprises at least one indicating electrode and at least one reference electrode. The reader is configured to operably engage with the disposable probe and provide pH information of a sample. The system is constructed and arranged to provide the pH information based on potentiometric measurement of the sample solution including a measurement of at least two signals. A first signal is received from the at least one indicating electrode when the at least one indicating electrode is in contact with the sample. A second signal is received from the at least one reference electrode when the at least one reference electrode is in contact with a reference solution. The system may include one or more components manufactured in a MEMS or other automated process, such as a disposable probe or probe portion manufactured in a MEMS process.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a top view of an embodiment of a pH sensor.

FIG. 2 illustrates a cross-sectional view of the pH sensor of FIG. 1 along A-A′ illustrated in FIG. 1.

FIG. 3 illustrates a cross-sectional view of another embodiment of a pH sensor.

FIG. 4 illustrates a cross-sectional view of another embodiment of a pH sensor.

FIG. 5 illustrates a top view of a measurement system comprising a disposable probe and a reader.

FIG. 5a illustrates a top view of the system of FIG. 5 with the indicating electrode assembly detached.

FIG. 5b is a perspective view of an indicating electrode assembly.

FIG. 6 illustrates a top view of a measurement system comprising a dual probe sensing assembly and a reader.

FIG. 7 illustrates a top view of a measurement system comprising a disposable probe and a reader.

FIG. 7a illustrates an enlarged view of the distal portion of the disposable probe of FIG. 7.

FIG. 7b illustrates an end view of the distal portion of the disposable probe of FIG. 7a after opening of an orifice.

FIG. 7c illustrates an enlarged view of the distal end of disposable probe of FIG. 7 after release of reference solution onto reference electrode.

FIG. 8 illustrates a top view of a measurement system comprising a disposable probe and a reader.

FIG. 8a illustrates a sectional view of the system of FIG. 8.

FIG. 8b illustrates a series of operational steps of the disposable probe of FIG. 8.

FIG. 9 illustrates a sectional view of a measurement system comprising a disposable probe and reader.

FIG. 10 illustrates a top view of an electrode assembly.

FIG. 10a illustrates a series of manufacturing step of the electrode assembly of FIG. 10.

FIG. 11 illustrates a top view of a multiple use disposable probe.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

As used herein and in the appended claims, the singular forms “a,” “an”, and “the” include plural references unless the content clearly dictates otherwise. Thus, for example, reference to “a bubble” includes a plurality of such bubbles and equivalents thereof known to those skilled in the art, and so forth, and reference to “the bubble” is a reference to one or more such bubbles and equivalents thereof known to those skilled in the art, and so forth.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “attached”, “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

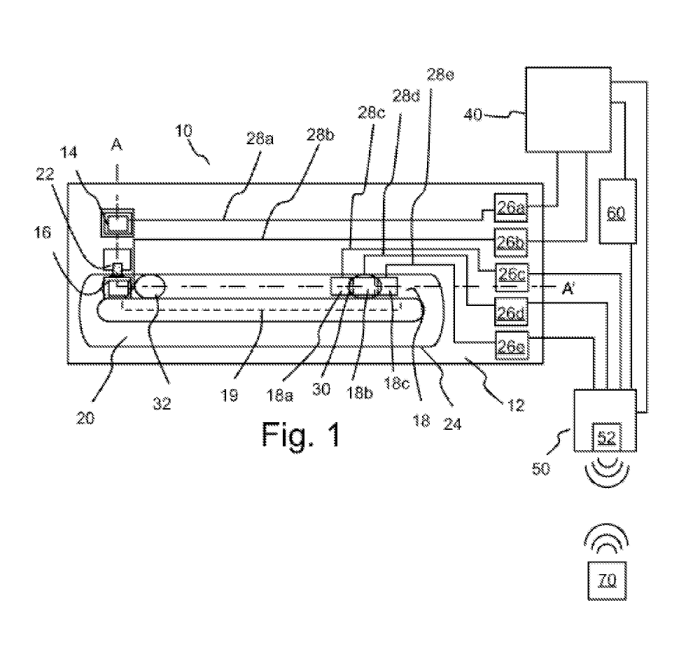

FIG. 1 illustrates a top view of a pH sensor 10 according to various embodiments which is readily formed to microscale or smaller (for example, nanoscale) dimensions, such as a pH sensor described in International Application Serial Number PCT/US2010/045847, the contents of which are incorporated herein by reference in its entirety. The term “microscale” as used in connection with the pH sensor hereof refers to sensors having dimensions smaller than one centimeter. In a number of embodiments, the dimensions of the pH sensors hereof are amenable to micro- and/or nanofabrication techniques. In a number of embodiments, the reference electrolyte solution volume was 20 cubic mm or less.

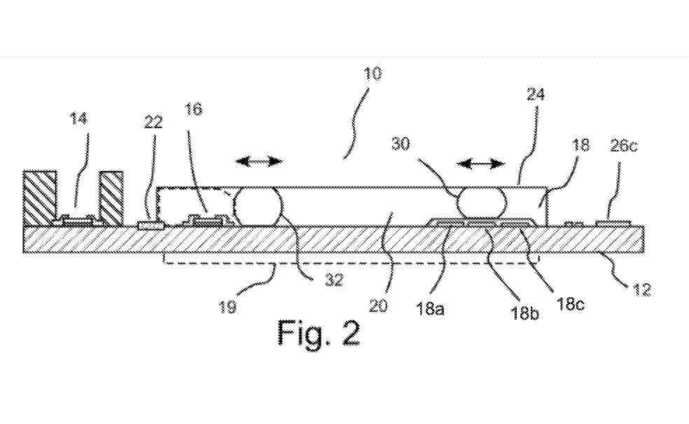

FIG. 2 illustrates a cross-sectional view of pH sensor 10 (taken along line A-A′ illustrated in FIG. 1). In a number of embodiments. pH sensor 10 can, for example, be formed in a size and configuration which allows for its implantation into a body (that is, within a human or animal) using a minimally invasive technique. In a number of embodiments, the length, width and height of pH sensor 10 are each less than 1 centimeter. Such a microscale pH sensor 10 may, for example, be used in a variety of applications to measure the pH of a sample electrolyte, a sample tissue, etc. Such applications include medical applications where the microscale pH sensor 10 is utilized to measure the pH of myocardial tissue, brain tissue, liver tissue, kidney tissue, lung tissue, etc.

In the representative embodiment of FIGS. 1 and 2, pH sensor 10 includes a substrate 12, a first electrode 14, a second electrode 16, a system for transporting a bubble 18, a fluidic closed loop channel 20, a liquid junction 22, a cover 24, a plurality of connection pads 26a through 26e, and a plurality of conductors 28a through 28e (see FIG. 1).

Substrate 12 may, for example, include any suitable type of material that is, for example, amenable to fabrication of the various electrodes and other layers that it supports. Suitable materials include, for example, silicon-based materials (for example, silicon, glass etc.), non-silicon-based materials, polymeric materials (for example, polydimethylsiloxane or PDMS) and other materials. In the case the sensor is to be implantable within a body, the material can, for example, be bio-compatible. In a number of embodiments, for example, substrate 12 is a glass substrate. The first electrode 14 functions as an indicating or sensing electrode, and may, for example, include any suitable type of material. In general, it is desirable that the material for first electrode 14 exhibit a wide pH response range, high sensitivity, fast response time, low potential drift, in sensitivity to stirring, a wide temperature operating range and a wide operating pressure range.

First electrode 14 can, for example, include an ion-selective field effect transistor (ISFET) or a metal oxide electrode. An ISFET is part of a solid-state integrated circuit. The ISFET exhibits a fast response time (on the order of 1 millisecond) and is quite rugged in in-vivo applications.

In the case of a metal oxide electrode, a number of metal oxides are suitable for use in first electrode 14. Metal oxides can, for example, be deposited upon a conductive (for example, metallic) layer that is deposited or formed on substrate 12. A metal oxide film or layer (for example, iridium oxide) can, for example, be created via a variety of techniques including electrochemical oxidation via potential cycling, reactive sputtering, anodic electrodeposition, thermal oxidation and others. In a number of embodiments, first electrode 14 includes platinum and iridium oxide. For such embodiments, the platinum can be deposited on the substrate 12, and the iridium oxide can be formed or deposited on the platinum. According to other embodiments, the first electrode 14 includes chromium and iridium oxide. For such embodiments, the chromium can be formed on the substrate 12, and the iridium oxide can be formed on the chromium. According to other embodiments, the first electrode 14 includes titanium and iridium oxide. For such embodiments, the titanium can be formed on the substrate 12, and the iridium oxide can be formed on the titanium. The first electrode 14 is positioned so that it comes into contact with the sample solution/electrolyte (for example, within a sample tissue) of which the pH is to be measured.

Second electrode 16 functions as a reference electrode, and may include any suitable type of material. Desirably, reference electrode 16 maintains a constant or substantially constant potential in the electrolyte solution. In a number of embodiments, second electrode 16 includes platinum and silver. For such embodiments, the platinum can, for example, be formed or deposited on substrate 12, and the silver can be formed or deposited on the platinum. According to other embodiments, second electrode 16 includes platinum and silver chloride. For such embodiments, the platinum can, for example, be formed or deposited on substrate 12, and the silver chloride can be formed or deposited on the platinum. According to other embodiments, second electrode 16 includes chromium and silver. For such embodiments, the chromium can, for example, be formed or deposited on the substrate 12, and the silver can formed on the chromium. According to other embodiments, second electrode 16 includes chromium and silver chloride. For such embodiments, the chromium can, for example, be formed or deposited on substrate 12, and the silver chloride can be formed on the chromium. According to other embodiments, second electrode 16 includes titanium and silver. For such embodiments, the titanium can, for example, be formed or deposited on substrate 12, and the silver can be formed on the titanium. According to other embodiments, second electrode 16 includes titanium and silver chloride. For such embodiments, the titanium can, for example, be formed or deposited on the substrate 12, and the silver chloride can be formed or deposited on the titanium. Second electrode 16 is positioned so that it is in contact with a reference solution within fluidic closed loop channel 20.

Bubble transport system 18 and bubbles 30 and 32 operate in connection with liquid junction 22 and the reference analyte solution within fluidic channel 20 as a fluidic switch or controller 19. Fluidic switch 19 is, for example, operable to place pH sensor 10 in an on state or in an off state. Fluidic switch 19 may be any type of fluidic switch suitable to provide a barrier between a fluid transporting member such as liquid junction 22 and the reference electrolyte solution. In a number of embodiments, fluid switch 19 is operable to turn pH sensor 10 (or another device) off and on by, for example, disrupting the ionic electrical connection between the analyte solution and the reference solution. Fluid switch 19 can also be operable to reduce or eliminate mass transfer between the analyte solution and the reference solution.

In a number of embodiments, as described in more detail hereinafter, bubble transport system 18 can, for example, use electrowetting-on-dielectric principles to effect switching functionality. According to various embodiments, bubble transport system 18 can, for example, include a plurality of electrodes. In the illustrated embodiment, bubble transport system 18 includes three electrodes 18a, 18b and 18c. Bubble transport system 18 may include any suitable type of material. In various embodiments, bubble transport system 18 includes platinum, an insulating layer (e.g., silicon oxide, parylene, etc.), and a hydrophobic layer (e.g., a fluorocarbon hydrophobic layer). In such embodiments, the platinum can, for example, be formed or deposited on substrate 12, and the insulating layer and the hydrophobic layer can be formed or deposited on the platinum. According to other embodiments, bubble transport system 18 includes chromium, an insulating layer, and a hydrophobic layer. For such embodiments, the chromium can, for example, be formed or deposited on substrate 12, and the insulating layer and the hydrophobic layer can be formed or deposited on the chromium. Bubble transport system 18 is positioned so that it is in direct contact with the reference solution of fluidic closed loop channel 20.

In the representative embodiment of FIGS. 1 and 2, fluidic channel 20 is a closed loop channel 20 which is collectively defined by substrate 12 and cover 24. Fluid closed loop channel 20 can, for example, include any suitable type of ionically conductive aqueous solution. For example, according to various embodiments, fluidic channel 20 includes a saturated potassium chloride solution. According to other embodiments fluidic channel 20 includes a saturated silver chloride solution. Fluidic channels hereof need not be closed loop fluid channels. The fluidic channels enable movement of one or more bubbles or, in the case where a bubble is generated within the channel as described below, the fluidic channel allows displacement of the liquid so that the one or more bubbles can be formed to a desired volume.

As shown in FIG. 1, in a number of embodiments, fluidic closed loop channel 20 surrounds bubble transport system 18, and includes a first bubble 30 and a second bubble 32. First bubble 30 is hydrodynamically connected to the second bubble 32 via the saturated reference solution. Thus, when first bubble 30 is driven from a first position to a second position, second bubble 32 moves from a third position to a fourth position. The third position is shown in solid lines in FIGS. 1 and 2, while the fourth position is show in dashed lines in FIGS. 1 and 2. Accordingly, first bubble 30 may be considered a “master” bubble and second bubble 32 may be considered a “slave” bubble. First and second bubbles 30 and 32 may, for example, include any suitable type of fluid material immiscible in the reference solution. At least bubble 32 can, for example, be immiscible in the analyte solution. For example, according to various embodiments, first and second bubbles 30 and 32 may include air, oil, a gas other than air (for example, hydrogen, oxygen, a mixture of oxygen and hydrogen, etc), etc.

As used herein, the term “bubble” refers to a globule or volume of one substance (a fluid) in another fluid (the reference electrolyte solution). A bubble can, for example, be formed of a gas that is immiscible in the liquid within channel 20 (that is, the saturated reference solution) or a liquid that is immiscible in the liquid within channel 20.

Liquid junction 22 is positioned between the sample or analyte electrolyte solution and the reference solution enclosed in fluidic closed loop channel 20 (for example, saturated potassium chloride), and provides for ionic electrical connection between the analyte electrolyte solution and the reference solution in fluidic closed loop channel 20. In a number of embodiments, liquid junction 22 is a member through which fluid transport can occur and may, for example, include a porous or permeable material. For example, according to various embodiments, liquid junction 22 includes a hydrophilic porous polymer. A porous material for liquid junction 22 can, for example, have a pore size of less than one micrometer. In a number of embodiments, liquid junction 22 is designed to limit or minimize mass exchange between the solution in the fluidic closed loop channel 20 and the sample electrolyte solution (for example, by limiting pore size in the case of a porous material). As shown in FIG. 2, liquid junction 22 is positioned between the substrate 12 and the cover 24 in the illustrated embodiment.

Cover 24 is connected to substrate 12, and cooperates with substrate 12 to define fluidic closed loop channel 20. Cover 24 may, for example, include any suitable type of impermeable material. In the case of an implantable pH sensor 10, cover 24 (and other components of pH sensor 10 which contact an organism) can, for example, be biocompatible. For example, according to various embodiments, cover 24 includes glass or polydimethylsiloxane. Cover 24 may be connected to the substrate 12 in any suitable manner. For example, according to various embodiments, the cover 24 is bonded to the substrate 12. In several embodiment in which cover 24 is glass and substrate 12 is PDMS, cover 24 is readily bonded to substrate 12 by simply pressing them together after 0₂ plasma treatment of surfaces. In, for example, cases in which the fluidic channel 20 width is relatively large (for example, about 1 mm or larger) an adhesive can be used to bond cover 24 to substrate 12.

As described above, in the illustrated representative embodiment of FIGS. 1 and 2, a plurality of connection elements or pads 26a through 26e are connected to substrate 12, and may include any suitable type of conductor. For example, according to various embodiments, connection pads 26a-e include platinum. According to other embodiments, connection pads 26a-e include chromium. According to other embodiments, connection pads 26a-e include titanium. According to other embodiments, connection pads 26a-e include gold. Connection pad 26a is connected to the first electrode 14 via conductor 28a. Connection pad 26b is connected to second electrode 16 via conductor 28b. Connection pads 26c, 26d and 26e are connected to electrodes 18a, 18b and 18c of bubble transport system 18 via the conductors 28c, 28d and 28e, respectively. Connection pads 26a-e provide for electrical connection of first electrode 14, second electrode 16, and electrodes 18a, 18b and 18c of fluidic switch 18 to one or more circuits external to the pH sensor 10. As illustrated in FIG. 1, first electrode 14 and second electrode 16 can, for example, be connected to measurement electronics or circuitry 40 which can, for example, include potentiometer circuitry as known in the art. Electrodes 18a, 18b and 18c of bubble transport system 18 can, for example, be in electrical connection with control electronics or circuitry 50.

The plurality of conductors 28a-e may, for example, be formed on a surface of substrate 12, and function to connect first electrode 14, second electrode 16, and electrodes 18a-c to respective connection pads 26a-e. As shown in FIG. 1 and as described above, a first conductor 28a connects first electrode 14 to first connection pad 26a, and a second conductor 28b connects second electrode 16 to second connection pad 26b. Similarly, individual conductors 28c-e connect electrodes 18a-c of bubble transport system 18 to corresponding connection pads 26c-e, respectively. Conductors 28a-e may, for example, include any suitable type of conductive material. For example, according to various embodiments, conductors 28a-e include platinum. According to other embodiments, conductors 28a-e include chromium. According to other embodiments, conductors 28a-e include titanium. According to other embodiments, conductors 28a-e include, for example, gold, copper, or aluminum.

In operation of the representative embodiment illustrated in FIGS. 1 and 2, first electrode 14 is exposed to the sample electrolyte (or to a sample tissue). When pH sensor 10 is in an off state (via fluidic switch 19), first bubble 30 is positioned on the “leftmost” (in the orientation the figures) electrode 18a of bubble transport system 18, and second bubble 32 is positioned against liquid junction 22. The positioning of the first bubble 30 and second bubbles 32 may, for example, be realized in any suitable manner. For example, according to various embodiments, electrowetting-on-dielectric techniques may be utilized to move the first bubble 30 and second bubble 32 to the respective positions. For such embodiments, the sequential activation of “rightmost” electrode 18c and “middle” electrode 18b of bubble transport system 18 may be utilized to cause first bubble 30 and second bubble 32 to move to the respective positions associated with the off state of pH sensor 10.

In the off state position, second bubble 32 can, for example, form a barrier over second electrode 16 and liquid junction 22, effectively blocking the fluid/electrical (ionic) connection between the sample electrolyte and the saturated solution in the fluidic closed loop channel 20, thereby reducing or preventing the dissolution of second electrode 16 into the saturated solution, and reducing or preventing mass exchange through liquid junction 22. When second bubble 32 is in the above-described, off-state position, immiscible phase interfaces (for example, gas-liquid or liquid-liquid immiscible interfaces) are formed between second bubble 32 and the sample electrolyte in or at the surface of the pores of liquid junction 22. The interfacial tension between the phases, for example, between a gas and the liquid phase) operates to reduce or block leakage of the sample electrolyte into fluidic closed loop channel 20. Maintaining pH sensor 10 in an off state extends the useful life of pH sensor 10 as compared to a sensor continuously maintained in an on state.

When a pH level is to be measured, pH sensor 10 is switched to an on state. To be switched to the on state, second bubble 32 is moved so that it does not form a barrier over second electrode 16 and the liquid junction 22, and thereby allows for the establishment of an electrical connection between the sample electrolyte and the saturated solution in fluidic closed loop channel 20. According to various embodiments, second bubble 32, which is hydrodynamically connected to first bubble 30, is moved away from second electrode 16 and liquid junction 22 by moving first bubble 30 away from “leftmost” electrode 18a of bubble transport system 18.

First bubble 30 may be moved away from “leftmost” electrode 18a of bubble transport system 18 in any suitable manner. For example, according to various embodiments, electrowetting-on-dielectric principles are utilized to move first bubble 30, which in turn causes movement of second bubble 32. In electrowetting-on-dielectric devices or systems, bubbles are transported by programming and sequentially activating arrays of electrodes.

For such embodiments, the activation of “leftmost” electrode 18a of bubble transport system 18 operates to move first bubble 30 away from “leftmost” electrode 18a of bubble transport system 18 and towards “rightmost” electrode 18c of bubble transport system 18. The movement of first bubble 30 towards the “rightmost” electrode 18c of bubble transport system 18 causes second bubble 32 to move away from second electrode 16 and liquid junction 22, thereby removing the barrier over second electrode 16 and liquid junction 22. The removal of the barrier allows for the establishment of the fluid/electrical (ionic) connection between the sample electrolyte and the saturated solution in fluidic closed loop channel 20.

In the manner described above, pH sensor 10 can be quickly switched between the off and on states, with very low energy consumption. By forming a barrier over second electrode 16 and liquid junction 22 during the off state, and exposing second electrode 16 and liquid barrier 22 to the saturated reference solution of the fluidic closed loop channel 20 only during the on state, dissolution of the second electrode 16 and mass exchange through the liquid junction 22 is reduced or minimized, thereby increasing the useful life of pH sensor 10.

As illustrated schematically in FIG. 1, at least one power source 60 such as a battery can be provided in electrical connection with sensor electronics 40 and control electronics 50. Power source 60 can, for example be used to power sensor electronics 40, control electronics 50 and bubble transport system 18 in the embodiment of FIG. 1. In a number of embodiments, pH sensor 10 can, for example, be actuatable and/or controllable via an external device 70 which communicates (for example, wirelessly via, for example, a radio frequency or RF signal) with, for example, a transceiver 52 in communicative connection with control electronics 50. Control electronics 50 can, for example, be programmed (for example, via one or more programmed processors) to cause bubbles 30 and 32 to move as describe above to enable pH sensor 10 to measure pH at some predetermined time cycle and/or in response to an external signal (for example, external to a body in which pH sensor 10 is implanted). When pH sensor 10 is activated or enabled, a pH reading is acquired by sensor electronics 40. Sensor electronics 40 is in communicative connection with control electronics 50 which effects control of bubble transport system 18. Once a measurement is obtained, pH sensor 10 can be placed in the off state or inactivated via control of bubble transport system 18 as described above. The measured pH value can, for example, be made available for use (for example, either for transmission to outside the body via transceiver 52, or for use by another implanted system, which can, for example, include a treatment device).

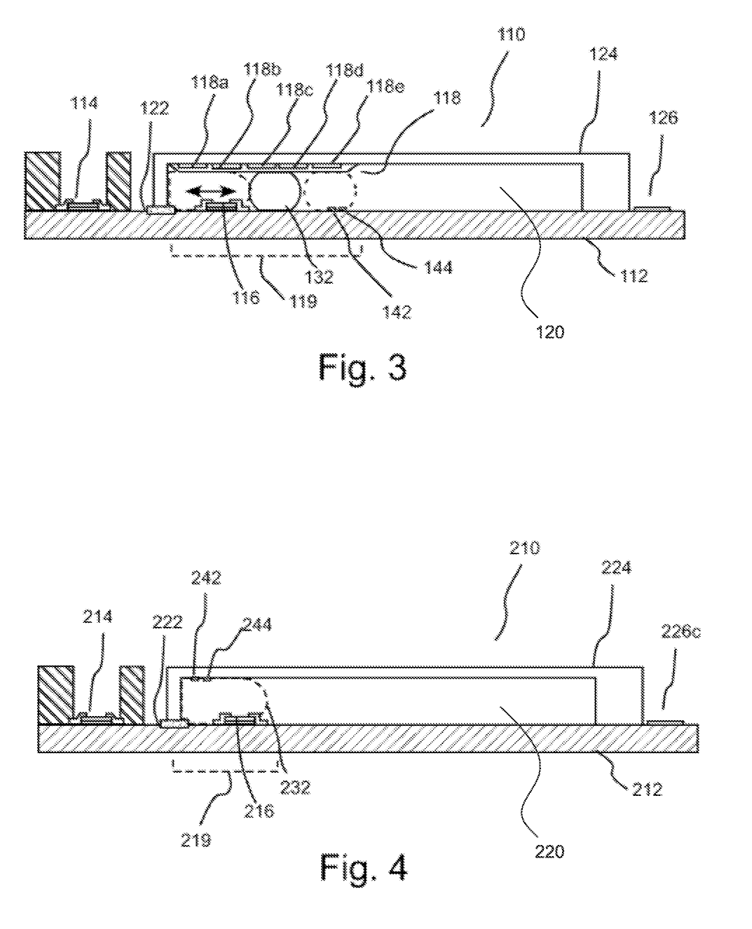

In several embodiments of the present invention, a pH sensor includes a single bubble to effect switching between an on state and an off state. For example FIG. 3 illustrates another representative embodiment of a pH sensor 110 in which a single bubble 132 within a channel 120 (formed between a cover 124 and a substrate 112) is used to form a barrier over a second or reference electrode 116 and a liquid junction 122 (as described in connection with pH sensor 10) to operate as a fluidic switch or controller 119. As described above, by forming or creating a barrier covering liquid junction 22, an off-state is created, wherein ionic electrical connection between the analyte solution (which contacts first or indicating electrode 114) and the reference solution within channel 120 is disrupted or prevented. Furthermore, in the off-state, bubble 132 reduces, minimizes or eliminates mass transfer between the analyte solution and the reference solution. The off state further reduces dissolution of electrode 116 within the reference solution. If bubble 132 is of sufficient size to cover electrode 116, dissolution (mass transfer) between electrode 116 and the reference solution can be further reduced, minimized or eliminated. Dissolution of second electrode 116 and mass exchange through the liquid junction 122 thus occurs to a significant extent only during the on state, thereby increasing the useful life of pH sensor 110.

In the embodiment of FIG. 3, gas bubble 132, which is a mixture of oxygen and hydrogen, is generated via electrolysis using an anode 142 and a cathode 144 that are positioned relatively close to each other (for example, within approximately 4 um in several embodiments). In the embodiment illustrated in FIG. 3, bubble transport system 118 (for example, an electrowetting-on-dielectric system) is positioned on a top surface of fluidic channel 120. Bubble transport system 118, can, for example, include an array of electrodes 118a-e, which are positioned on an inner surface of cover 124 (that is, on a top surface of fluidic channel 120). In the illustrated embodiment, the electrolysis electrodes used to create bubble 132 (that is, anode 142 and cathode 144) are placed on substrate 112. To create bubble 132 (or a plurality of bubbles as, for example, discussed in connection with pH sensor 10 of FIGS. 1 and 2), one can, for example, apply a potential difference of approximately 5 V between and anode/cathode pair such as anode 142 and cathode 144.

In operation of fluidic switch 119, bubble 132 is first generated via electrolysis using anode 142 and cathode 144 (see rightmost dashed lines in fluidic channel 120). The size of the bubble created can, for example, be controlled via control of the time that a potential is applied. To place fluid switch 119 in an off state, bubble 132 is transported via bubble transportation system 118 to cover liquid junction 122 (see leftmost dashed lines in fluidic channel 120) and, in several embodiments, to cover reference electrode 116. To place fluid switch in an on state, bubble 132 is transported via bubble transportation system 118 so that is does not cover either liquid junction 122 or reference electrode 116.

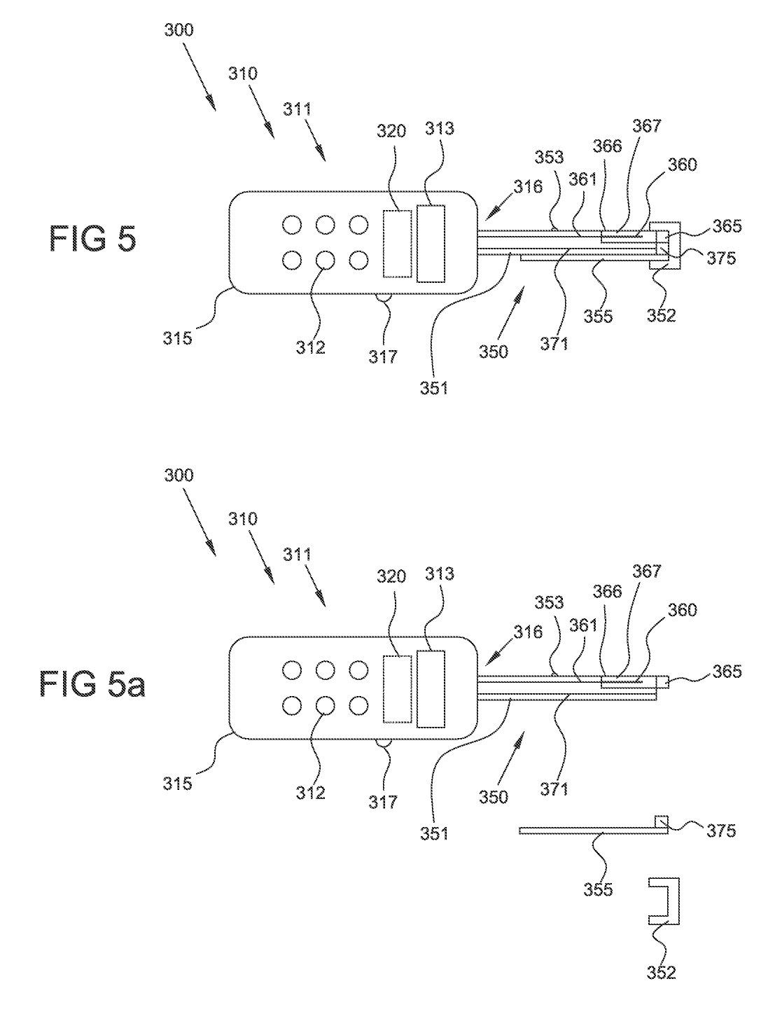

FIG. 4 illustrates another representative embodiment of a pH sensor 210 in which a single bubble 232 within a channel 220 (formed between a cover 224 and a substrate 212) is used to form a barrier over a second or reference electrode 216 and a liquid junction 222, as described in connection with pH sensors 10 (of FIGS. 1 and 2) and 110 (of FIG. 3), to operate as a fluidic switch 219. As described above, by forming or creating a barrier covering liquid junction 222, an off-state is created, wherein ionic electrical connection between the analyte solution (which contacts first or indicating electrode 214) and the reference solution within channel 220 is disrupted or prevented. If bubble 232 is of sufficient size to cover electrode 216, dissolution (mass transfer) between electrode 216 and the reference solution can be further reduced, minimized or eliminated.

In operation of fluidic switch 219, bubble 232 is first generated via electrolysis using anode 242 and cathode 244 (see rightmost dashed lines in fluidic channel 220). As described above, the size of the bubble created can, for example, be controlled via control of the time that a potential is applied. To place fluid switch 219 in an off state, bubble 232 is generated to a size to cover liquid junction 222 and, in several embodiments, to cover reference electrode 216. To subsequently place fluid switch in an on state, bubble 232 is reduced in size or completely eliminated via reversing of the electrolysis process using anode 242 and cathode 244 so that it does not cover either liquid junction 222 or reference electrode 216. To effect bubble reduction or elimination, catalysis can be used to lower the energy barrier in the reverse process. For the case of bubble 232 including hydrogen and oxygen bubble, platinum (Pt) can, for example, be used as a catalyst. In a number of embodiments, anode 242 and cathode 244 can, for example, be made to include a catalytic material such as Pt. When an electric potential is applied to the anode 242 and cathode 244, bubble 232 grows. When the electric potential is shut off, bubble 232 shrinks. In an alternative embodiment, a source of a catalyst such as Pt can be provided separately from anode 242 and cathode 244.

Fluidic switches or controller such as fluidic switches or controllers 19 (of FIGS. 1 and 2), 119 (of FIG. 3) and 219 can, for example, be used in other devices where it is desirable to control fluid connection, ionic conduction and/or mass transfer across a member though which a fluid can be transported (for example, a porous or permeable member such as a porous polymeric member, a permeable membrane etc).

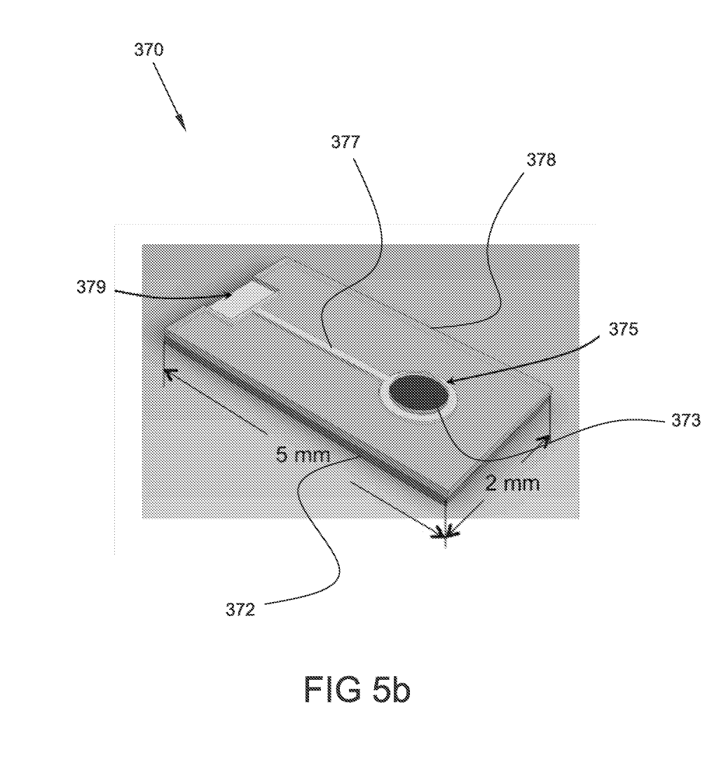

FIG. 5 illustrates an embodiment of a system for taking pH readings using a handheld device and a sensing probe, according to embodiments of the present invention. System 300 comprises reader 310, and a disposable probe 350. In a typical configuration, operation of system 300 is based on potentiometric measurement of pH using an indicating electrode (e.g. an iridium oxide electrode) that is compared to a known electric potential generated by a reference electrode (e.g. a silver-silver chloride electrode) in contact with a reference solution. One or more components of system 300 are typically pre-conditioned to produce a known output, such as to avoid a calibration step, in manufacturing and/or at time of use. Reader 310 comprises housing 315 into which is integrated user interface 311. User interface 311 includes display 313 and buttons 312. Display 313, typically a liquid crystal or touch screen display, may display measured pH readings, as well as system and other information. System information may include but is not limited to: system readiness information; power levels; alert or alarm condition information; current status of disposable; and combinations of these. Other information provided by system 300 via user interface 311 may include environmental conditions such as temperature, humidity and/or pressure information recorded by sensor 317 of reader 310 and/or sensor 353 of probe 350.

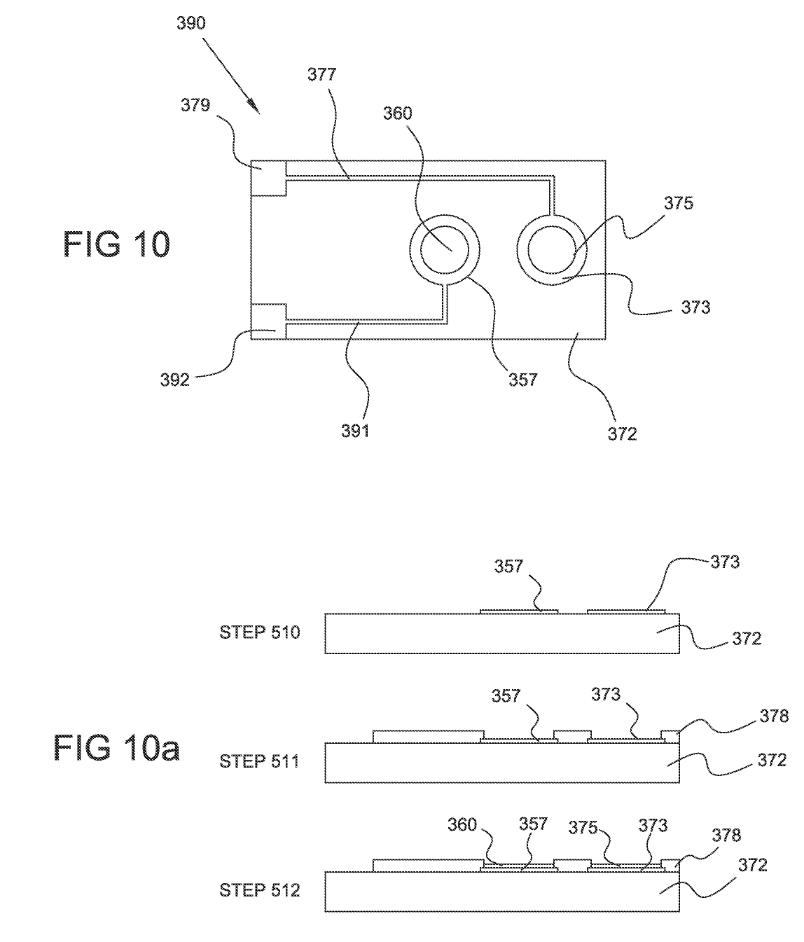

Housing 315 further includes an electromechanical port, port 316, configured to operably engage with the proximal end of disposable probe 350. Probe 350 comprises housing 351, indicating electrode 375, and liquid junction 365. Liquid junction 365 is typically constructed and arranged as is described above in reference to FIGS. 1 through 4. Indicating electrode 375 comprises at least one indicating electrode, which may be activatable, such as being surrounded by a removable cover. The removable cover, not shown, may be a manually or automatically removable cover. In one embodiment, the removable cover comprises a controllable orifice. Controllable orifices may be configured to provide a controllable opening, such as the manipulatable membranes and other controllable orifices as is described in applicants co-pending application, Provisional Application Ser. No. 61/531,238, entitled MEASUREMENT DEVICE WITH SENSOR ARRAY, by Clark et al, filed of even date herewith, the disclosure of which is incorporated herein by reference in its entirety. In a typical embodiment, electrode 375 is manufactured in a MEMS fabrication process. In one embodiment, electrode 375 is manufactured as is described in reference to FIG. 10a herebelow.

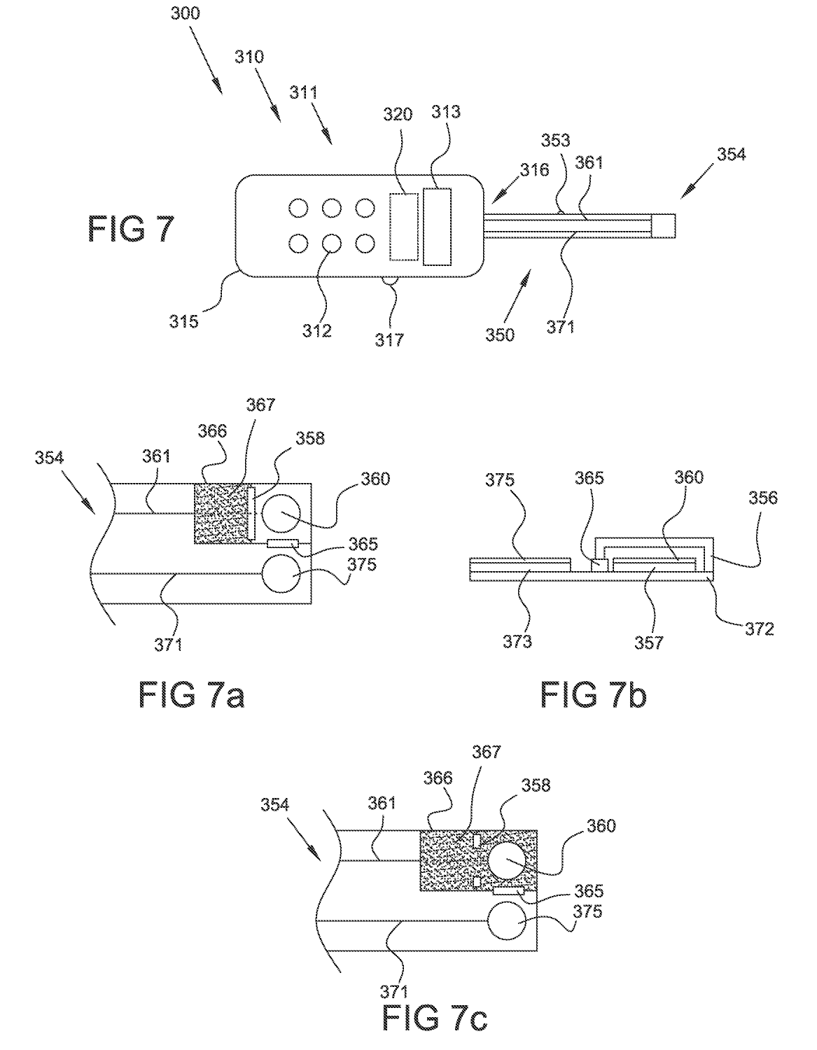

Probe 350 also comprises reservoir 366, which houses reference solution 367 and reference electrode 360. Reference electrode 360 may be a silver/silver-chloride reference electrode, or another known reference source configured with predictable pH sensitivity in the presence of a known reference solution. Alternatively, reference electrode 360 may be an iridium oxide electrode as is described in reference to FIG. 7 herebelow. Reference solution 367 may be a KCl solution, or other suitable solution to be used in cooperation with reference electrode 360.

Wires 361 and 371 travel through housing 351 to port 316 such as to electrically connect reference electrode 360 and indicating electrode 375, respectively, to electronics module 320. Module 320, contained within housing 315 of reader 310, is configured to determine pH levels based on electrical signals received on wires 361 and 371 and to display pH information on display 313. Module 320 may be further configured to interpret other information such as signals received from sensor 353 and/or sensor 317. Module 320 may be further configured to store data, communicate with one or more external devices (e.g. via wired or wireless communications), perform internal diagnostic checks, and the like. Stored data may include but is not limited to: reference electrode 360 information; indicating electrode 375 information; storage information; information on number of available uses remaining; date of manufacture, date of expiration, and other date information; and combinations of these. Liquid junction 365, when saturated with reference solution 367, allows electrical connection between indicating electrode 375 and reference electrode 360 when probe 350 is submersed in a sample solution.

Probe 350 may include a removable cap, cap 352, typically a plastic material that attaches to the distal tip of probe 350 and which is removed prior to testing of a sample solution. Cap 352 may be configured to prevent liquid junction 365 from drying out (e.g. during storage or between uses) or otherwise to protect the distal portion of probe 350.

Reference electrode 360 is typically constructed and arranged to be used to perform multiple sample measurements. Indicating electrode 375 is typically constructed and arranged to be used in a single sample measurement, after which it is replaced with an unused indicating electrode 375. Indicating electrode 375 is attached to handle 355 to facilitate easy removal of handle 355 from probe 350, as is shown in FIG. 5a (FIG. 5a also showing cap 352 having been removed from the distal end of probe 350).

Reader 310 may include one or more additional components, not shown but selected from the group consisting of: a chamber configured to store multiple probes 350; a reservoir configured to store reference solution 367; and combinations of these.

Referring to FIG. 5b, a perspective view of an indicating electrode assembly is illustrated, according to embodiments of the present invention. Indicating electrode assembly 370 comprises substrate 372 and may be configured in the approximate dimensions shown, but typically in a smaller configuration. Substrate shapes may be circular, square, rectangular, or other geometric configurations. Substrate 372 typically comprises one or more materials selected from the group consisting of: glass; silicon; and plastic. Trace 377, typically a conductive strip such as a titanium strip, is fixedly attached to substrate 372 and electrically connects electrical connecting pad 379 and indicating electrode 375. Trace 377 may be connected to an electrical mounting pad 373 onto which indicating electrode 375 is fixedly attached. Mounting pad 373 is typically incorporated to facilitate the mounting of indicating electrode 375 to substrate 372, such as when indicating electrode 375 comprises an iridium oxide electrode created using an electro-deposition process. Additional or alternative processes for electrode manufacture include but are not limited to: sputtering; evaporation; oxidation (e.g. of layers); and combinations of these. Another layer 378, typically comprising silicon dioxide, is fixedly attached to substrate 372 and acts as a protective and/or insulating layer and includes an opening which surrounds indicating electrode 375. Connecting pad 379 is positioned to allow an electrical connection to another component of the system, such as a conductor configured to transmit electrical signals, such as wire 371 of FIG. 5.

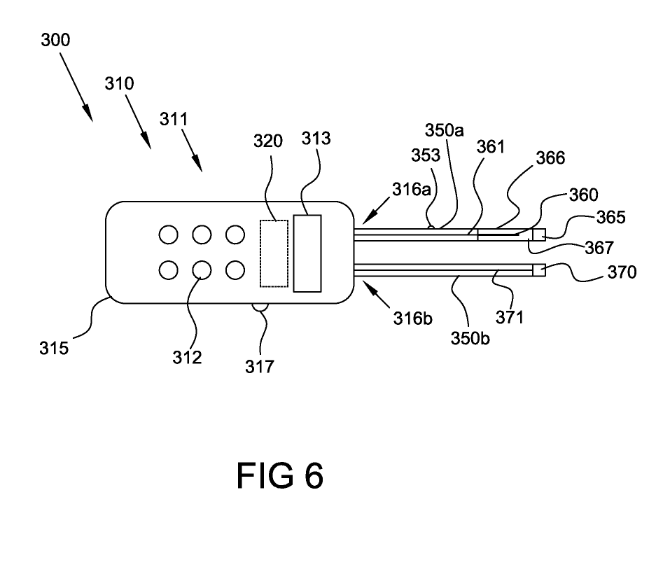

FIG. 6 illustrates an embodiment of a system for taking pH readings using a handheld device and a dual probe sensing assembly, according to embodiments of the present invention. System 300 includes reader 310 and a dual probe assembly comprising probe 350a and 350b. Reader 310 comprises housing 315, user interface 311 including buttons 312 and display 313, sensor 317 and electronics module 320, each typically of similar construction and configuration to the similar components of reader 310 of FIG. 5.

Reader 310 further includes an electromechanical port 316a configured to operably attach to probe 350a, such as to transmit electrical signals to or from electronics module 320. Reader 310 further includes an electromechanical port 316b configured to operably attach to probe 350b, such as to transmit electrical signals to or from electronics module 320.

Probe 350a comprises reference electrode 360, reservoir 366, reference solution 367, liquid junction 365, and wire 361, each typically of similar construction and configuration to the similar components of probe 350 of FIG. 5. Probe 350a is configured for multiple uses, such as multiple uses within a pre-determined period of time (e.g. 6 months) and/or a fixed number of measurements.

Probe 350b comprises an indicating electrode 375 and wire 371, typically of similar construction and configuration to the similar components of probe 350 of FIG. 5. Probe 350b is typically configured for single use, such that multiple probes 350b are used with a single probe 350a. Alternatively, probe 350b is configured for multiple uses, such as by including multiple indicating electrodes 375, such as are described in reference to applicant's co-pending application, Provisional Application Ser. No. 61/531,238, entitled MEASUREMENT DEVICE WITH SENSOR ARRAY, by Clark et al, filed of even date herewith, the disclosure of which is incorporated herein by reference in its entirety.

FIG. 7 illustrates an embodiment of a system for taking pH readings using a handheld device and a disposable probe comprising a releasable reference solution, according to embodiments of the present invention. System 300 includes reader 310 and probe 350. Reader 310 comprises housing 315, user interface 311 including buttons 312 and display 313, sensor 317, electronics module 320 and port 316, each typically of similar construction and configuration to the similar components of reader 310 of FIG. 5.

Probe 350 comprises distal portion 354 with components described herebelow in reference to FIGS. 7a and 7b. Probe 350 may include one or more sensors, such as sensor 353, typically configured to measure one or more system or environmental parameters such as temperature, humidity and/or pressure. Wires 361 and 371 travel through probe 350 to port 316 such as to electrically connect one or more reference electrodes and/or one or more indicating electrodes positioned on probe distal end 354 to electronics module 320. In this embodiment, distal portion 354 is disposable, or all of probe 350 is disposable including distal portion 354. This is different from, e.g., the system of FIG. 5 in which only the indicating electrode is disposed after use.

Referring additionally to FIGS. 7a and 7b, top and end views of distal portion 354 are shown. Reference electrode 360 and indicating electrode 375 comprise iridium oxide electrodes, typically created in an electro-deposition process, such as is used in a MEMS fabrication process. Reference electrode 360 and indicating electrode 375 may be fixed to titanium pads, pads 357 and 373, respectively where pads are mounted on substrate 372. Chamber 356 surrounds reference electrode 360. Liquid junction 365, positioned along a wall of chamber 356, fluidly connects reference electrode 360 to indicating electrode 375 (e.g. when probe 350 is submersed in a test solution as has been described hereabove).

Reservoir 366 contains reference solution 367 and is fluidly separated from chamber 356 by a controllable orifice, barrier 358. Barrier 358 is configured to be manipulated, such as to cause one or more cracks or other openings. Manipulations can be performed by an operator (e.g. applying a breaking or crushing force to barrier 358) or by a component of system 300 (e.g. a hydraulic or pneumatic piston, a solenoid driven piston, or other force applying mechanism controllably actuated by electronic module 320). As shown in FIG. 7c, openings in barrier 358 allow reference solution 367 to enter chamber 356, cover reference electrode 360 and saturate liquid junction 365. Numerous controllable orifices can be used in addition to or in place of barrier 358, including but not limited to: sliding walls or doors; valves, such as electrical, pneumatic or hydraulic valves; membranes that rupture, such as via heat activation; and combinations of these. A conductor, wire 361, is electrically connected to reference electrode 360 (e.g. via pad 357), and is configured to transmit electrical signals to electronics module 320. A second conductor, wire 371, is electrically connected to indicating electrode 375 (e.g. via pad 373), and is also configured to transmit electrical signals to electronics module 320. Additional wires are typically included, not shown but connecting to one or more functional elements of disposable probe 350 such as heat elements, membranes, valves, sensors, and the like.

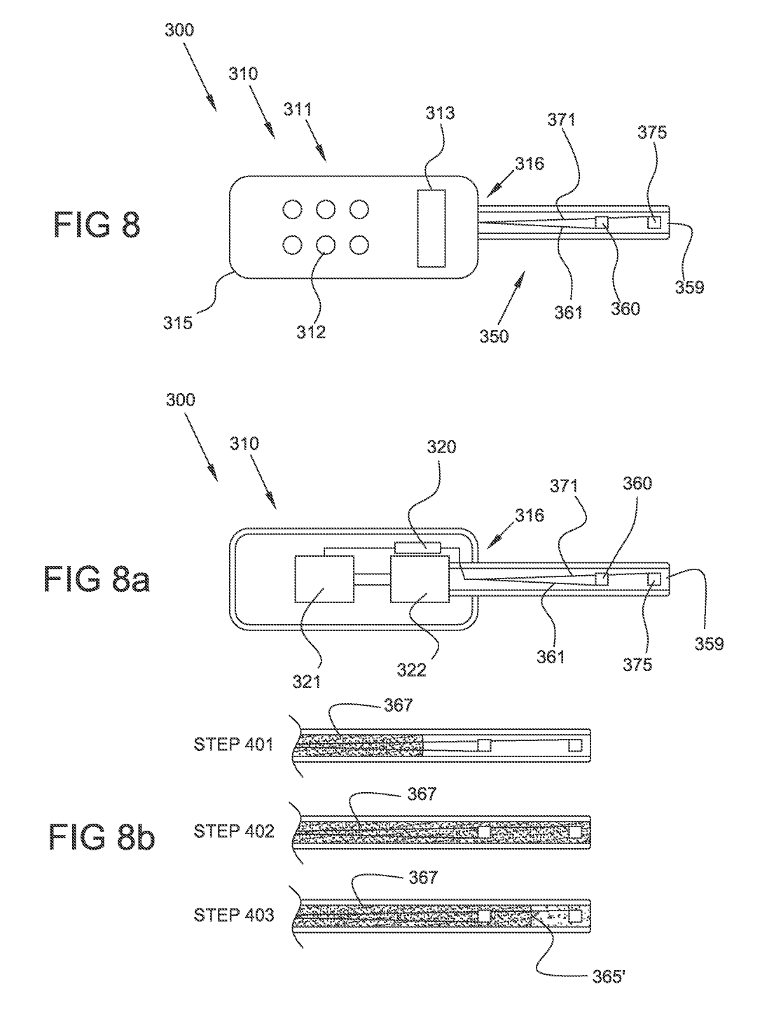

FIG. 8 illustrates an embodiment of a system for taking pH readings using a handheld device and a probe comprising a pump and fluidic channel, according to embodiments of the present invention. System 300 includes reader 310 and probe 350. Reader 310 comprises housing 315 and user interface 311, including buttons 312 and display 313, each typically of similar construction and configuration to the similar components of reader 310 of FIG. 5.

Reader 310 further includes an electromechanical port 316 configured to operably attach to probe 350, such as to transmit electrical signals to or from an electronics module. Port 316 may be configured to electrically connect to additional electrical components of disposable probe 350, such as heat elements, membranes, valves, sensors, and the like, all not shown but described herein. Probe 350 includes a channel for transporting fluids to a distal portion of probe 350, fluid channel 359. Port 316 is further configured to fluidly connect fluid channel 359 to an internal fluid channel of reader 310. Port 350 may include other fluid connecting means, not shown.

Probe 350 further comprises reference electrode 360 and indicating electrode 375, each positioned within fluidic channel 359. Indicating electrode 375 is positioned distal to reference electrode 360 such that sample solution can be drawn into fluidic channel 359 while a reference solution (e.g. reference solution 367 shown in FIG. 8b) is in contact with reference electrode 360, as is described in detail herebelow. Wire 361 is electrically attached to reference electrode 360, and wire 371 is electrically attached to indicating electrode 375.

Referring now to FIG. 8a, pump 321, reservoir 322 and electronics module 320, each of reader 310, are illustrated. Electronics module 320 is electrically connected to wires 361 and 371 via port 316. Electronics module 320 is electrically connected to pump 321, such that fluid can be moved to or from reservoir 322. Fluid exiting reservoir 322 enters fluidic channel 359 via port 316. Fluid entering reservoir 322 is delivered from fluidic channel 359, also via port 316.

Referring now to FIG. 8b, a series of activation steps are illustrated, in which fluid is transported, in two directions, within the fluidic channel 359. In STEP 401, pump 321 has pumped reference solution 367 into the fluidic channel 359, but without reaching reference electrode 360. No sample solution has been brought in contact with indicating electrode 375. In STEP 402, reference solution is further pumped (to the right of the page as shown) such that reference electrode has filled fluidic channel 359 to the distal end of probe 350, and both reference electrode 360 and indicating electrode 375 are covered with reference solution 367. In STEP 403, the distal tip of probe 350 has been placed into a sample solution. Pump 321 has been operated to cause a reversal of flow (compared to STEPS 401 and 402) such that sample solution has been drawn into a distal portion of fluidic channel 359. Pumping is performed such that indicating electrode 375 is covered by the sample solution and reference electrode 360 remains covered by reference solution 367. Within fluidic channel 359, a virtual liquid junction 365′ is formed at the interface between the sample solution and reference solution 367.

By “virtual liquid junction,” we mean direct contact between two different liquids such that ionic electrical connection is achieved without the need for liquid junction through a porous material. In essence, the conventional porous material is virtual and the direct liquid junction is achieved.

In another embodiment, reverse pumping can be avoided by opening the end of the channel around the indicating electrode. In that embodiment, the reference liquid is pumped to the end of channel (STEPS 401′, 402′). Then probe is then lowered into a test solution (STEP 403′) so that a virtual liquid junction is formed between the two liquids at the end of the channel between the two electrodes.

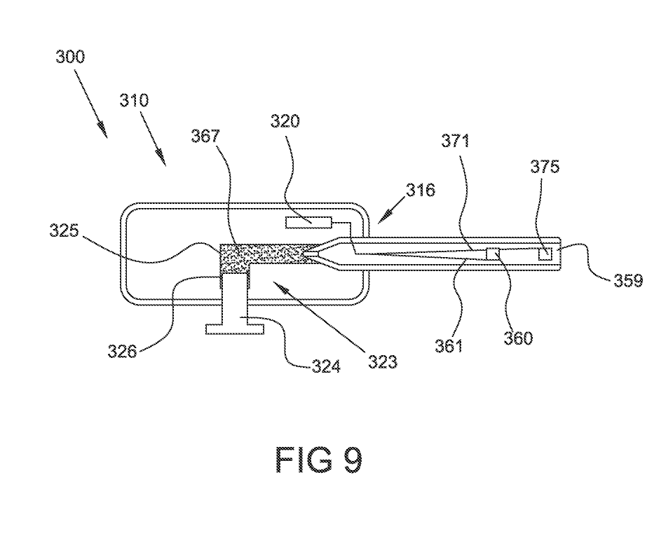

FIG. 9 illustrates an embodiment of a system for taking pH readings using a handheld device and a probe comprising a manual pumping mechanism and fluidic channel, according to embodiments of the present invention. System 300 is similar to system 300 of FIG. 8a other than pump 321 has been replaced with pumping mechanism 323. Pumping mechanism 323 comprises plunger 324 which operably translates within fluidic channel 325. Fluidic channel 325 is fluidly connected to fluidic channel 359 via port 316. An o-ring 326 creates a fluid seal between plunger 324 and fluidic channel 325 such that depressing plunger 324 causes fluid to flow in fluidic channel 359 in a direction toward the right side of the page as shown. Retraction of plunger 324 causes fluid to flow in fluidic channel 359 in a direction toward the left side of the page as shown. Alternative to or in addition to plunger 324, other pumping or fluid transport mechanisms may be included. In one embodiment, mechanical stops may be positioned along a fluidic channel to allow specific amounts of fluid to be displaced, such as to specifically fill channels or specifically position volumes of fluids or fluid boundaries (e.g. a create or move a virtual liquid junction).

FIG. 10 illustrates a top view of an electrode assembly, according to embodiments of the present invention. Electrode assembly 390 comprises substrate 372. Electrode assembly 390 comprises reference electrode 360 and indicating electrode 375, both mounted to substrate 372. Trace 377, typically a conductive strip such as a titanium strip, is fixedly attached to substrate 372 and electrically connects electrical connecting pad 379 and indicating electrode 375. Trace 377 may be connected to an electrical mounting pad 373 onto which indicating electrode 375 is fixedly attached. Mounting pad 373 is typically incorporated to facilitate the mounting of indicating electrode 375 to substrate 372, such as when indicating electrode 375 comprises an iridium oxide electrode created using electrochemical oxidation via potential cycling, reactive sputtering, anodic electrodeposition, thermal oxidation, etc. Trace 391, typically a conductive strip such as a titanium strip, is fixedly attached to substrate 372 and electrically connects electrical connecting pad 392 and reference electrode 360. Trace 391 may be connected to an electrical mounting pad 357 onto which reference electrode 360 is fixedly attached. Mounting pad 357 is typically incorporated to facilitate the mounting of reference electrode 360 to substrate 372, such as when reference electrode 360 comprises an iridium oxide electrode created using electrochemical oxidation via potential cycling, reactive sputtering, anodic electrodeposition, thermal oxidation, etc.

Electrode assembly 390 is typically fabricated using electro-deposition and other MEMS fabrication processes as is described in reference to FIG. 10a herebelow. In an alternative embodiment, multiple indicating electrodes 375 are mounted to substrate 372 such that the single reference electrode 360 is used to perform multiple readings with the multiple indicating electrodes 375.

FIG. 10a illustrates a step-wise process of manufacturing an assembly comprising an indicating electrode and a reference electrode mounted to a substrate, according to embodiments of the present invention. In the illustrated embodiment, STEP 510 shows substrate 372 with titanium mounting pads 357 and 373. Titanium mounting pads 357 and 373 have been mounted to substrate 372 using a deposition process, creating a layer on substrate 372, such as titanium, for example using an E-beam evaporator. Curing and etching processes have removed portions of the deposited layer, leaving mounting electrodes 373 as shown.

STEP 511 shows the assembly of STEP 510, including layer 378, for example a layer comprising SiO₂. Layer 378 has been deposited onto substrate 372 using a deposition process, followed by a curing and etching process to achieve the configuration shown. Layer 378 may comprise a passivation layer used to protect and/or insulate electrical traces and other components from damage or other adverse effects, such as when exposed to sample and/or reference solutions.

STEP 512 shows the assembly of STEP 511, now including indicating electrode 375 and reference electrode 360. Indicating electrode 375 and/or reference electrode 360 can comprise an Iridium Oxide pad that has been deposited directly onto a mounting electrode 373 and/or 357, respectively. After deposition of the indicating electrode 375 and reference electrode 360, a post fabrication process has been performed, that can include a thermal treatment followed by a voltage treatment such as a voltage treatment comprising application of a known voltage to the iridium oxide layer in the presence of a buffer solution. The voltage is applied for a fixed period of time, in constant or varied levels, in order to modify the chemical composition of the iridium oxide layer. When exposed to a sample solution, this voltage modification can be used to cause the indicating electrode 375 to produce a known voltage response relative to the pH of the reference solution, such as to avoid calibration.

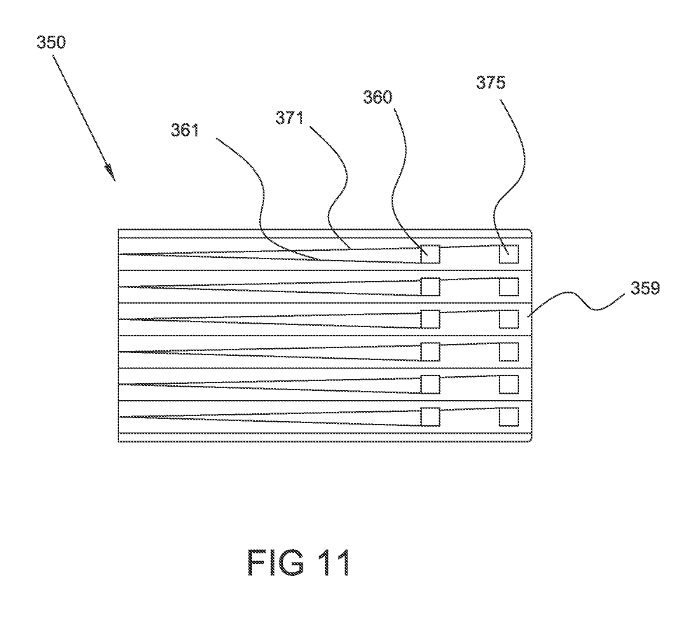

FIG. 11 illustrates a top view of a probe with multiple fluidic channels, according to embodiments of the present invention. Probe 350 comprises multiple reference electrodes 360 and multiple indicating electrodes 375. Wires 361 and 371 are electrically attached to reference electrodes 360 and indicating electrodes 375, respectively, such as to connect to an electronics module, as has been described in detail hereabove. Multiple fluidic channels 359 provide a fluid pathway between the proximal and distal ends of probe 350. Channels 359 may be configured to attach to a two-way pumping mechanism, as has been described in detail hereabove. Each indicating electrode 375 and reference electrode 360 is positioned within a fluidic channel 359. Probe 350 is configured to be inserted into a reader, such as reader 310 of FIGS. 8 and 9. One or more pumping mechanisms fluidly connect with channels 359 to transport reference fluid and sample solution within one or more portions of channel 359. A reader may be configured with a single insertion location, with multiple ports aligned with the multiple fluidic channels 359. Alternatively, a reader may include a single translatable port that moves (vertically as shown on the page) to independently and controllably fluidly connect with the multiple fluidic channels 359.

The multiple fluidic channels 359 may be in fluid communication with one or more reservoirs and/or one or more pumping mechanisms (reservoirs and pumping mechanisms not shown but described in reference to FIGS. 8 and 9 hereabove. In one embodiment, a single reservoir is in fluid communication with multiple fluidic channels. In an alternative embodiment, a first reservoir is in communication with a first fluidic channel and a second reservoir is in fluidic communication with a second fluidic channel. In one embodiment, a single pump moves fluid within multiple fluidic channels. In an alternative embodiment, each fluidic channel comprises a separate pump configured to move fluid within the respective channel, for example, a first pump moves fluid within a first fluidic channel and a second pump moves fluid within a second fluidic channel and so on.

The foregoing description and accompanying drawings set forth a number of examples of representative embodiments at the present time. Various modifications, additions and alternative designs will become apparent to those skilled in the art in light of the foregoing teachings without departing from the spirit hereof, or exceeding the scope hereof, which is indicated by the following claims rather than by the foregoing description. All changes and variations that fall within the meaning and range of equivalency of the claims are to be embraced within their scope.

What is claimed is:

1. A system for obtaining a pH measurement comprising:
at least one disposable probe comprising:
at least two electrodes; and
at least one fluidic channel arranged and constructed to support a virtual liquid junction between a reference solution and a sample,
wherein at least one electrode is in contact with the reference solution and function as a reference electrode and at least on electrode is in contact with the sample and functions as an indicating electrode.

2-8. (canceled)

9. The system of claim 1 further comprising a cap configured to prevent drying out of the virtual liquid junction, or to engage with an end of the disposable probe and to protect said end.

10-13. (canceled)

14. The system of claim 1 wherein the at least one reference electrode and the at least one indicating electrode is positioned within the at least one fluidic channel.

15. The system of claim 1 wherein the at least one fluidic channel is constructed and arranged to transport the reference solution.

16. The system of claim 15 further comprising a pumping assembly constructed and arranged to cause said transport.

17. The system of claim 1 wherein the at least one fluidic channel is constructed and arranged to transport the sample.

18-23. (canceled)

24. The system of claim 1 further comprising a reservoir constructed and arranged to store reference solution.

25. The system of claim 24 further comprising a barrier positioned between the at least one reference electrode and said reference solution reservoir, the barrier configured to be removed, broken, or opened to allow flow of fluid.

26-29. (canceled)

30. The system of claim 1 wherein the disposable probe comprises a first portion and a second portion, and wherein said first portion comprises the at least one reference electrode and said second portion comprises the at least one indicating electrode.

31-35. (canceled)

36. The system of claim 1 wherein the disposable probe comprises a multi-layer construction comprising at least one substrate.

37-40. (canceled)

41. The system of claim 1 wherein at least one electrode comprises an iridium oxide electrode.

42-50. (canceled)

51. The system of claim 1 wherein the at least one reference electrode is mounted on at least one substrate.

52. The system of claim 1 wherein the at least one indicating electrode is manufactured using a MEMS process.

53-72. (canceled)

73. The system of claim 1 wherein the reader comprises an electronics module constructed and arranged to perform at least one of storing data; communicating with at least one external device via wired or wireless communications; performing internal diagnostic checks; and combinations thereof.

74-91. (canceled)

92. The system of claim 1 further comprising a reader configured to operably engage with the disposable probe and provide pH information of the sample.

93. The system of claim 15, wherein the at least one fluidic channel is constructed and arranged to transport the reference solution to the at least one reference electrode.

94. The system of claim 1 further comprising multiple fluidic channels, wherein a first fluidic channel comprises a first indicating electrode and a first reference electrode, and a second fluidic channel comprises a second indicating electrode and a second reference electrode.

95. The system of claim 25 wherein the barrier is constructed and arranged to be operator activated.

96. The system of claim 1 further comprising a removable cover surrounding the at least one electrode.

97. The system of claim 1 further comprising an additional disposable probe.
